(19) United States
(12) Patent Application Publication
(10) Pub. No.: US 2012/0158008 A1
(43) Pub. Date: Jun. 21, 2012

(54) WOUND DRAINAGE DEVICE AND SYSTEM

(76) Inventor: Rolando GARCIA, Golden Beach, FL (US)

(21) Appl. No.: 12/968,442

(22) Filed: Dec. 15, 2010

(51) Int. Cl. A61B 17/34

(52) U.S. Cl. 606/108

(57) ABSTRACT

An insertion device has pivotable elongated members manipulable by a hand, one of which holds a trocar, and the other a target through which the trocar will pass as the elongated members are pivoted. A tube is connected to the trocar or elongated member holding the trocar, and the tube is passed through body tissue from a position interior to the body to a position exterior to the body, together with the trocar. An end of the tube is positioned in a fluid accumulating area of the body, and passes to a container external to the body. The container contains markings which enable a determination of the quantity of passed fluid. At intervals, the quantity of fluid is determined, and the container is replaced with another container.

### FIELD OF THE INVENTION

The invention relates to surgical instruments and related systems for draining fluid from wounds.

### BACKGROUND OF THE INVENTION

Surgical procedures can cause excess fluid to accumulate in the body. If left untreated, the accumulation of excess fluid may cause pain and increase the risk of infection of the surrounding tissue. Even so called “minimally or less” invasive procedures can lead to fluid accumulation. As a result, it is often desirable to provide a way for this fluid to drain out of the body, such as by inserting a drainage tube into the treated area to drain any excess fluids that may otherwise accumulate.

Insertion of a drainage tube into the treated area typically involves use of a trocar or other piercing object having a sharp point for piercing through tissue. The drainage tube is connected to the rearward portion of the trocar in a manner that, once the tube is in a desired position, it may then be removed or separated from the trocar. The trocar is then discarded and other components of the drain, such as fluid reservoirs, anchors, and the like, are then added to the drain assembly.

Previously, the procedure of inserting a drainage tube required the surgeon to manipulate the trocar from multiple locations and manually navigate it through a surgical incision toward a fluid sac. Then, the surgeon would guide the trocar back toward the outer surface of the body. Once the sharp tip of the trocar pierced the outer surface of the patient's skin, the surgeon would grip it and pull the remainder of the trocar and a portion of the tubing out of the body.

This manual procedure can be time consuming, difficult to perform, and potentially dangerous for the surgeon and assisting medical staff. For example the process of gripping a rearward part of the trocar to first push it through, and then gripping a portion near the sharp forward tip to pull it through required the surgeon to reposition their grip on the trocar, which can be both time-consuming and difficult to perform successfully. It also means that the physician would not know precisely where to expect the trocar to pierce the outer surface of the skin, thereby making the process inefficient and potentially more dangerous.

One reason why this process is inefficient is because it requires some steps of assembly of the drain to be delayed until others are fully completed. For instance, an anchor often is used to help hold the drainage tube in place. The anchor typically surrounds the tubing and is attached to a portion of the patient's skin where the trocar and tubing exit the body. Not knowing precisely where this exit would be meant that the anchor could not be added until after the trocar and portion of tubing had exited the body. The anchor would then be guided onto the tubing and attached to the patient. Thus, the insertion of the drain was time consuming because positioning of the anchor could not begin until other steps were completed first.

In addition to the added time and complexity involved with past procedures, not knowing where the trocar will exit the body also increases the risk that the surgeon or assistants also may be punctured by the sharp tip of the trocar. Physicians incorrectly expecting the trocar to protrude through the patient's skin in one location, for example, may inadvertently puncture themselves or their assistants as the trocar exits the patient's body at a different location.

Once installed, traditional drains utilize a container where excess fluid is collected over time. Part of assessing a patient's recovery progress involves monitoring the amount of fluid that is collected by the drain. As a result, the container is removed from the drain periodically so that the fluid can be emptied into a measuring cup. The emptied container is then be reconnected to the drainage tube so that it can resume collecting fluid. This process continues until a determination is made that the drain may be removed, which often does not occur for a day or more.

Thus, the container of conventional drains may be reused several times over the course of treatment with medical personnel periodically removing the container to empty it, measuring fluid output, and then reattaching the container to the drainage tubing. This process of repeated detachment, emptying, and reattachment of the container, however, requires several time-consuming steps. Moreover, the amount of handling of the container and transfer of fluid increases the risk of contamination and disease transmission.

### SUMMARY OF THE INVENTION

The invention is generally directed toward insertion tools, installation kits, and related components for a wound drainage system.

One embodiment of the invention is directed to a surgical device having two elongated members that mutually rotate to open or close two respective ends away from, or toward, each other. One end of one elongated member has a trocar, advantageously curved, that is used to guide drainage tubing to a desired position in the patient's body, and then return out of the body. The other elongated member has an end that may be pressed against, or disposed near, the location where the surgeon desires the trocar to exit the body. Rotation of the elongated members toward a closed position causes the trocar to follow a path having a known exit point. Gripping surfaces, for example curved loops, advantageously with flattened and or knurled or roughened surfaces, may be provided on the elongated members, to allow better manipulation or control of the device.

In one embodiment of the invention, the drain insertion tool rotates about a fixed axis so that the ends of the elongated members follow a partially circular path of a predetermined radius of curvature. The curvature of the trocar may be similarly curved so that as it moves during closing and opening of the device the sharp tip of the trocar moves in relatively the same direction as its curved shape, facilitating penetration of the trocar through body tissue. In another embodiment, the elongated members rotate about a moving axis of rotation, such as by rotating over cammed surfaces, to provide a more complex pathway for the trocar to follow.

Another embodiment of the invention includes a surgical kit for installing a wound drain. In addition to having an insertion tool as described above, the kit advantageously also include a drainage tube, an anchor for the drainage tube, one or more fluid receptacles, suture, and the like. Some embodiments of the invention provide an agent (such as an antibiotic or disinfectant) on or within one or more components, and in other embodiments the fluid receptacles may be interchangeably replaced, so that requirements for handling of drained fluids, and the attendant risk of infection and or disease transmission, are reduced.

### BRIEF DESCRIPTION OF THE DRAWINGS

FIG. 1 is an illustration of one embodiment of a drain insertion device of the invention;

FIG. 2 is an illustration of the drain insertion device of FIG. 1 in use;

FIG. 3 is another illustration of the drain insertion device of FIG. 1 in use;

FIG. 4 is an illustration of an embodiment of an anchor that may be used with the invention;

FIG. 5 is an illustration of another embodiment of an anchor that may be used with the invention;

FIG. 6 is an illustration of a drain installation kit of the present invention;

FIG. 7 is an illustration of an embodiment of fluid containers of the invention.

### DETAILED DESCRIPTION OF THE INVENTION

The invention is directed toward improved devices and methods for inserting and maintaining a fluid drain in a patient. While several features and aspects of the invention will be discussed in detail below, the invention generally relates to an improved insertion tool and an installation kit having many components useful for inserting and maintaining the drain during use.

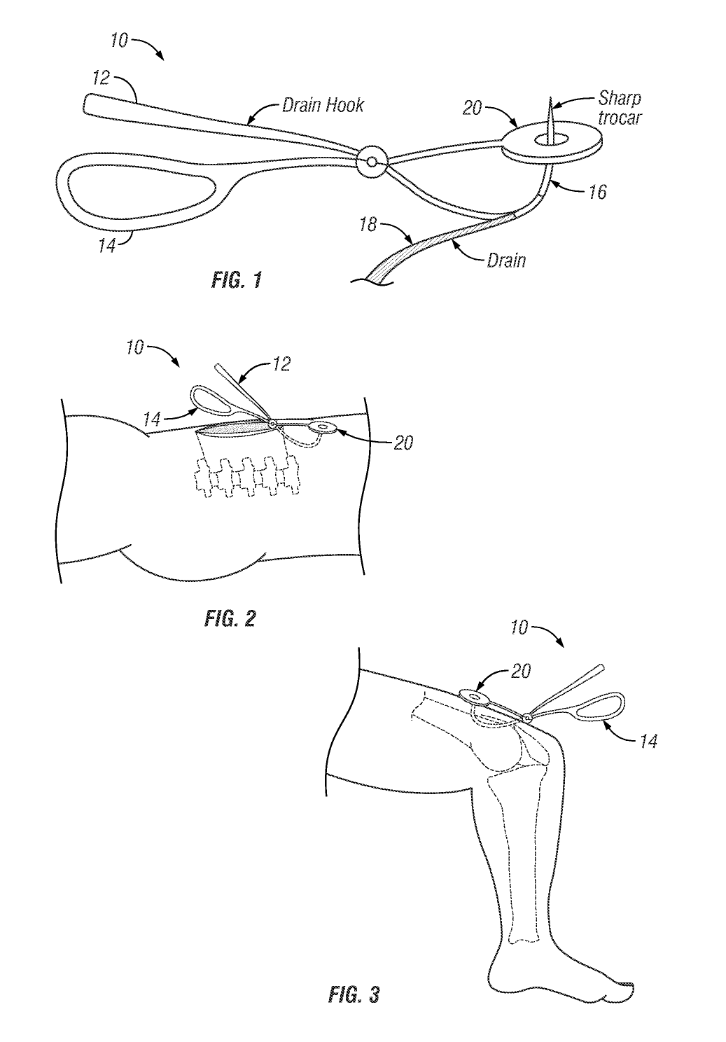

FIG. 1 shows an embodiment of an insertion tool 10 of the present invention. As shown the insertion tool 10 includes two arms or elongated members 12, 14 that are pivotally connected together. One elongated member 12 can be manipulated to guide a trocar 16 to its desired location. The other elongated member 14 may be used to position and align the location at which a piercing object, such as a needle, cannula, or trocar 16 exits the patient.

Referring again to FIG. 1, the elongated members 12, 14 may be manipulated in a manner similar to operation of a pair of scissors. Thus, the proximal ends of the elongated members may be configured for handling and manipulation by a human hand. For example, a proximal end of one or both elongated members may have a loop through which a physician may place one or more fingers or thumb in order to grip the device securely. Alternatively, one or both of the proximal ends of the elongated members may have an open gripping surface that may permit the user to hold it in a variety of positions and apply pressure urging the proximal ends of the elongated members to move toward each other. The gripping portions may additionally be provided with a knurled or roughened surface to improve a grip thereof.

A distal end 20 of one of the elongated members 14 may be configured to allow attachment and manipulation of a trocar 16, as by a clip, friction fit, set screw, threadable connection, tightenable loop, or other means as would be understood by one skilled in the art. As discussed above, the leading edge of the trocar 16 has a sharp point that allows it to pierce the patient's tissue near the area to be drained. As shown in FIG. 1, the trocar 16 may advantageously be curved to more easily penetrate tissue, particularly if the curve of the trocar is aligned to match an arc of curvature of the pivoted elongated members 12, 14. Alternatively stated, the curvature of the trocar 16 is advantageously selected so that, as the elongated members 12, 14 are closed toward each other, the path through which the trocar 16 moves corresponds at least generally to the curvature of the trocar 16 itself.

Thus, if the pivoting motion of the two elongated members 12, 14 is about a fixed axis of rotation, the curvature of at least the leading portion of the trocar 16 may have a similar radius of curvature as the circular path through which it travels. Alternatively, the rotational movement of one elongated member 12, 14 relative to the other may be more complex, such as by rotation about cammed surfaces that provide more complex path through which the trocar 16 moves. An example of this may be found in the four bar linkage of vice gripping pliers, or the known crank slider, truss, or five bar linkages, or a cam within a groove, the cam being provided on one elongated member, and the groove provided on another elongated member. This may allow, for instance, the trocar 16 to be more easily guided toward the fluid during an initial portion of closing movement of the elongated members 12, 14, while subsequently allowing the trocar 16 to return outward more easily during further closure of the elongated members 12, 14. In one embodiment, the trocar 16 is aligned more parallel to at least one of elongated members 12, 14 initially, and is then rotated due to the cammed configuration to turn more perpendicularly with respect to the elongated member in order to pass more directly outside of the body.

Regardless of the path through which the trocar 16 travels due to different configurations of the pivoting connection of the elongated members 12, 14, it is desired that the leading edge of the trocar 16 generally point in the direction of travel so that its sharp point may help navigate through the tissue. Thus, a tangent of the curved trocar near its leading edge preferably differs from the direction of travel by less than about 20 degrees, and more preferably differs by less than 10 degrees while the leading edge of the trocar is disposed within the patient's body.

The drainage tube 18 may be directly connected to a nearby portion of the trailing end of the trocar 16. For instance, the rearward portion or end of the trocar 16 may be configured so that the drainage tube 18 may be connected directly to it. For example, the rearward portion of the trocar 16 may have one or more outwardly extending protrusions or teeth over which the drainage tube 18 may be fitted. Alternatively, the drainage tube may be connected to elongated member 12, adjacent trocar 16, to follow the path of trocar 16 through body tissue. Similarly, clamping mechanisms may be used to hold the trocar 16 and drainage tube 18 together during insertion. Skilled artisans would appreciate that these examples are representative, but not limiting, and that other ways of connecting the drainage tube 18 and trocar 16 may be used without departing from the invention as described herein.

More particularly, the drainage tube 18 need not be directly connected to the trocar 16, but instead may be connected to the elongated member 12 that is associated with the trocar 16. In this manner, the trocar 16 may either be integrally formed with the elongated member 12, may be permanently connected to it, or may be removably associated with the elongated member 12. In turn, a portion of the distal end of the elongated member 12 may be configured to receive the drainage tubing 18. For example, a portion of the distal end of the elongated member 12 associated with the trocar 16 may have a partially hollowed portion or receptacle where an end of the drainage tube 18 may be connected. Preferably, the connection of the drainage tube 18 to the elongated member 12, trocar 16, or other associated component is such that the tube follows the pathway to the treated area being created by the trocar 16.

Returning again to FIG. 1, the distal end 20 of the second elongated member 14 may be configured such that it may be placed against the outer surface of the tissue and provide a visual indication, or target, of where the trocar 16 will exit the body. This may be accomplished, for instance, by providing a rounded end having an eye through which the trocar 16 will travel upon closing of the elongated members. The material that forms the distal end 20 of the second elongated member 14 may then be placed against the patient's skin at a location where it is desired that the trocar 16 exits the body.

The eye of the rounded end may be fully surrounded by material as illustrated, or alternatively may be only partially surrounded by material. For example, the distal end 20 may be open-ended, such as having a c-shape or u-shaped configuration. This open-ended configuration may be beneficial if the curvature of the portion of the trocar 16 that will pass through the eye or opening defined by the distal end 20 of the second elongated member 14 is different from the curved path through which this portion of the trocar 16 travels as the members are closed toward each other. In such cases, the open end may allow greater clearance for a portion of the trocar 16 as it passes through the distal end 20 of the second elongated member 14.

Distal end 20, if sufficiently rigid, advantageously additionally serves as a supporting member for holding body tissue in place during penetration of trocar 16, facilitating insertion of trocar 16 and drainage tube 18 through the body tissue.

Providing a predictable location through which the trocar 16 will exit the patient also allows an anchor 22 to be removably disposed on the distal end 20 of the second elongated member 14. In this manner, the step of threading the anchor onto the drainage tubing 18 can begin earlier and more efficiently than could be achieved in the past. The distal end 20 of the second elongated member 14 has a lower surface that may contact the patient's body and an upper surface that is more readily visible to the physician performing the procedure.

Anchor 22 and distal end 20 may be mutually adapted to remain in connection until trocar 16 and drainage tube 18 have been passed through anchor 22. Further, anchor 22 may be placed on, or secured to the target body tissue site, as by adhesive, suturing, stapling, or other known method, and the distal end of elongated member 14 then placed over or into connection with anchor 22. In this manner, it may be known not only that trocar 16 will pass through anchor 22, but also that anchor 22 is in a desired location.

More particularly, in an embodiment of the invention, anchor 22 may be associated with the distal end 20 of the second elongated member 14 by placing it in communication with the lower surface so that when the distal end 20 is pressed toward the patient's body it holds the anchor 22 in a desired position. Alternatively, the anchor 22 may be disposed on the outward facing side of the distal end 20 of the second elongated member 14. Once the anchor 22 is threaded onto the drainage tube 18, the distal end 20 of the second elongated member 14 may be moved out of its position in contact with the patient so that the anchor 22 may then be attached to the patient.

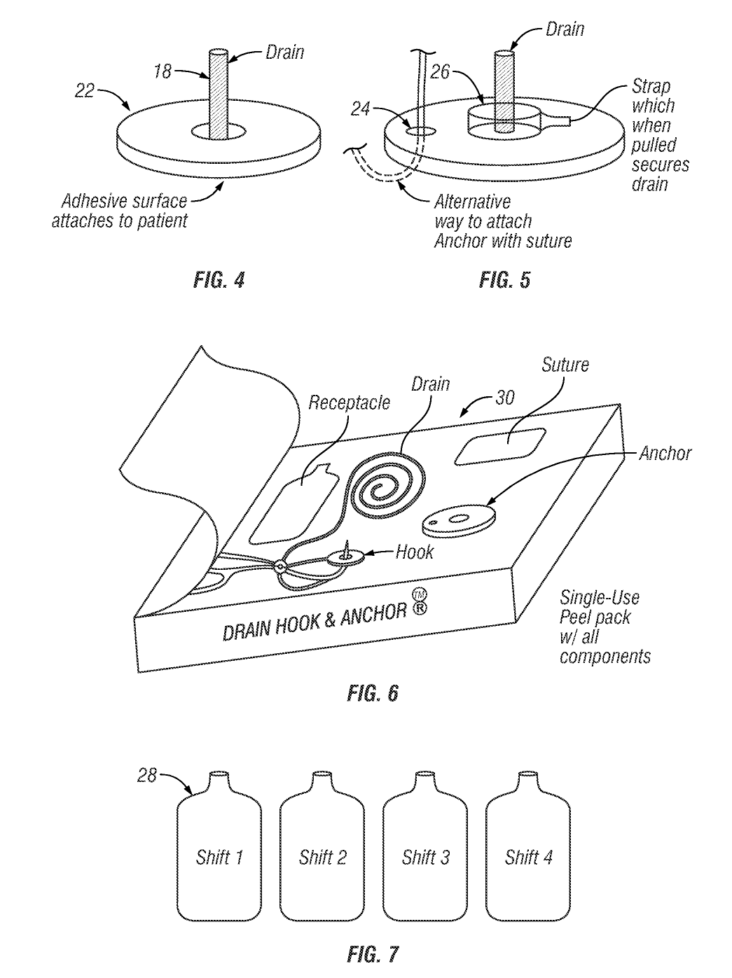

Referring to FIGS. 4 and 5, the anchor 22 may have an adhesive on at least a portion of the surface that contacts the patient. In addition, or as an alternative to using an adhesive, the anchor 22 may be configured with other ways of attaching it to the patient. For instance, FIG. 5 illustrates that the anchor 22 may be configured with one or more eyelets or recesses 24 through which a suture may be threaded. Skilled artisans having the benefit of this disclosure may recognize that still other alternatives for holding the anchor 22 in a desired position may be used as well.

The eye or opening of the anchor through which the drainage tube 18 is threaded may be configured to grip or hold the tube in place so that it resists inadvertent attempts to move or dislodge the drainage tube 18 relative to the anchor 22. One way this may be achieved is by making the opening or eye of the anchor 22 approximately the same size or slightly smaller than the outer perimeter of the drainage tube 18. This allows the anchor 22 to fit snugly around the drainage tube 18 and hold it in place with an interference fit.

Alternatively, the anchor 22 may not be configured with an opening or eyelet. Instead, the portion of the anchor 22 contacting the patient may be a sheet of material having no opening or eye until the trocar 16 forms one by piercing through it. In this embodiment, it may be beneficial for the outer perimeter of the trocar 16 to be smaller than that of the drainage tube 18 so that as the tube is pulled through the anchor 22 the pierced material may stretch and grip the tubing.

In yet another embodiment, the anchor 22 may be configured with other ways of gripping the drainage tube 18. As shown in FIG. 5, for instance, the anchor 22 may be configured with a strap 24 that can be tightened around the drainage tube 18 when desired. Other devices, such as clamps or clips, may also be used in conjunction with the anchor 22 to grip the drainage tube 18.

In use, the trocar 16 and drainage tube 18 are attached to the first elongated member 12 of the insertion device 10, while an anchor 22 is attached to the distal end 20 of the second elongated member 14. The trocar 16 is then advanced along a curved path and moved through the skin or tissue to a location near the wound that the surgeon believes may require draining. This may be done by gradually closing the elongated members 12, 14 while holding or pressing the distal end 20 of the second elongated member 14 at a location where the surgeon prefers the trocar 16 to exit the body.

Eventually, as the elongated members 12, 14 are closed further, the tip of the trocar 16 moves through the eye or opening of the distal end 20 of the second elongated member 14 as well as through the anchor 22. This continues until the proximal end of the drainage tube 18 also passes through the distal end 20 of the second elongated member 14 and the anchor 22. In this manner, the drainage tube 18 may be threaded onto the anchor 22 without having to wait until the insertion device 10 is removed.

Once the trocar 16 and a portion of the drainage tube 18 have exited the patient, further closing of the elongated members 12, 14 or advancement of the trocar 16 and drainage tube 18 by the insertion device 10 may not be needed. Instead, it may be preferable that the insertion device 10 be prevented from unintended re-opening of the elongated members 12, 14 while the anchor 22 is being attached to the patient. Thus, the elongated members 12, 14 may be configured such that once they are closed a predetermined distance or amount a ratchet or locking mechanism engages to prevent inadvertent reopening.

The ratchet or locking mechanism may be disposed near the pivoting connection between the first and second elongated members 12, 14, or may by disposed on proximal ends of the elongated members 12, 14 so that they engage when they are less than a predetermined distance apart. Preferably, any ratchet or locking mechanism does not engage until at least the leading edge of the trocar 16 has exited the patient.

The distal end of the drainage tube 18 generally resides in or near the wound, and the proximal end of the tube resides outside of the body. The tube is connected to the anchor 22 as previously described. The anchor 22 is fastened to the surface of the skin with an adhesive, or one or more sutures, for example. Once the tube 18 and anchor 22 are positioned, the inserter device 10 may be removed.

After the anchor 22 and drainage tube 18 are in their desired position, a receptacle 28 may be connected to the drainage tube to collect fluid from the wound. As discussed above, prior drains reused the same receptacle over the period of use of the drain. As a result, monitoring and maintenance of the drain was time-consuming and presented increased risks of contamination, disease transmission, and patient infection.

Referring to FIG. 7, the present invention reduces the time and risks of prior drains by providing a plurality of disposable receptacles 28. Each of the receptacles 28 of the invention is configured so that a health provider may easily measure the output of fluid without needing to transfer the fluid to a different container or receptacle.

As such, at least a portion of the receptacle 28 allows a health provider to view the fluid contained therein and compare it to a gauge that indicates fluid volume at multiple, graduated levels. For example, the receptacle may be formed of a low-cost transparent or translucent material such a plastic. Once a determination is made that the receptacle 28 is sufficiently full, it may be removed and properly discarded along with its contents, or used to store or transport the fluid if further analysis is desired. A second receptacle 28 is then connected to the drainage tube 18 to resume collecting fluid.

To reduce the likelihood of infection or disease transmission further, one or more components such as the drainage tubing 18, anchor 22, or receptacle 28 may be impregnated, coated, or filled at least in part, with an antibiotic, disinfectant, or similar agent or combination of agents. Preferably, the agent is present in sufficient quantity to function for at least half of the duration of the usage of the drain, and more preferably is present in sufficient quantities to last throughout the entire period of use of the drain.

Referring now to FIG. 6, two or more of the components described herein also may be combined to for a single use peel pack, including a bin and a releasably sealed surface layer, or surgical kit 30. Preferably, the kit 30 includes at least the insertion tool 10, trocar 16, drainage tube 18, anchor 22, and a plurality of holders or receptacles 28. Optionally, the kit 30 may also have suture for attaching the anchor to the patient or for other potential uses with the drain. Other components that may be provided include a drain exchange wire or drain unclogging wire.

Once used, the component parts of the kit may be discarded. As a result, it is preferable that the materials used for the insertion tool, receptacles, drainage tube, anchor, and other components are low cost. The component pieces of the kit 30 may be sterilized or assembled in a sterile environment and then sealed to prevent contamination during storage. When the surgeon is ready to install the drain, the protective packaging thereby helps minimize the risk of contamination without requiring the components to be sterilized when the kit is opened at surgery.

It will be appreciated by persons skilled in the art that the present invention is not limited to what has been particularly shown and described herein above. For example, although reference is made to surgery and surgeons, the present invention contemplates use by any suitable healthcare provider in other procedures, such as minimally invasive or less invasive procedures. In addition, unless mention was made above to the contrary, it should be noted that not all of the accompanying drawings are to scale. A variety of modifications and variations are possible in light of the above teachings without departing from the scope and spirit of the invention.

What is claimed is:

1. A surgical device for inserting a drainage tube, comprising:
a first elongated member having a proximal end including a gripping surface and a distal end including means to hold an attached trocar; and
a second elongated member pivotally connected to said first elongated member and having a proximal end including a gripping surface and a distal end having an alignment member defining a location through which a trocar held by said first elongated member will pass when said first and second elongated members are mutually pivoted;
wherein an attached trocar is passable through body tissue by mutually pivoting said first and second elongated members when body tissue is interposed between said attached trocar and said support member, whereby a drainage tube connected to the attached trocar or first elongated member is thereby passable through the body tissue.

2. The surgical device of claim 1, wherein rotation of the first elongated member relative to the second elongated member is about a predetermined axis of rotation.

3. The surgical device of claim 2, wherein the trocar has a radius of curvature corresponding to the radius of curvature through which the trocar passes when the first and second elongated members are rotated.

4. The surgical device of claim 1, wherein rotation of the first elongated member relative to the second elongated member is about a moving axis of rotation.

5. The surgical device of claim 4, wherein the first and second elongated members are pivotally connected with cams.

6. The surgical device of claim 1, wherein said support member comprises an anchor disposed on the distal end of the second elongated member, wherein relative rotation of the first and second elongated members cause the trocar to pass through a portion of the anchor.

7. The surgical device of claim 6, wherein the anchor has a contact surface on which an adhesive is applied.

8. The surgical device of claim 6, wherein the anchor further comprises an eyelet for receiving a suture.

9. The surgical device of claim 1, wherein the first elongated member comprises means for connecting a wound drainage tube thereto.

10. The surgical device of claim 1, wherein said alignment member further serves as a support member, operative to maintain a position of body tissue as the body tissue is penetrated by the trocar.

11. A surgical kit for installing a wound drainage system comprising:
a) a surgical device for inserting a drainage tube comprising:
a first elongated member having a proximal end including a gripping surface and a distal end including means to hold an attached trocar; and
a second elongated member pivotally connected to said first elongated member and having a proximal end including a gripping surface and a distal end having a visual indication member defining a location through which a trocar held by said first elongated member will pass when said first and second elongated members are mutually pivoted;
b) a drainage tube; and
c) an anchor having a contact surface operative to secure said anchor to the skin of a patient.

12. The surgical kit of claim 11, wherein at least one of the components of the kit is treated with an antibiotic.

13. The surgical kit of claim 11, further comprising a plurality of interchangeable fluid receptacles, and wherein the receptacles have graduated volume indicators.

14. The surgical kit of claim 11, wherein the distal end of the second elongated member is configured to receive the anchor, and wherein relative rotation of the first and second elongated members cause the trocar to pass through the anchor.

15. The surgical kit of claim 14, wherein the first elongated member or attached trocar is configured to connect to the drainage tube, and wherein further relative rotation of the first and second elongated members causes the drainage tube to pass through the anchor.

16. The surgical kit of claim 11, wherein the trocar has a radius of curvature corresponding to the radius of curvature through which the trocar passes when the first and second elongated members are mutually pivoted.

17. A device for installing a tube in a patient, comprising:
a first elongated member having a distal end including means for connecting a piercing object, and a proximal end adapted to be manipulated, said first elongated member passable into the body of the patient proximate an area to be drained;
a second elongated member having a distal end including extensions defining an opening, and a proximal end adapted to be manipulated, said opening positionable proximate an exterior portion of the body of the patient to be pierced;
a pivot defining a path and connecting said first and second elongated members, wherein a piercing object connected to said first elongated member passes through said opening when said first and second elongated members are pivoted about said pivot through a portion of said path;
whereby a tube connectable to said first elongated member distal end or the piercing object is passable through a portion of the path from a position proximate an area to be drained to a exterior portion of the body, whereby fluid may be drained from an area to be drained to the exterior of the body of the patient.

18. The device of claim 17, further including an anchor connectable to the tube and operative thereby to secure the tube to an exterior portion of the body.

19. The device of claim 18, wherein the anchor and said distal end of said second elongated member are adapted to be mutually connectable.

20. The device of claim 17, wherein said pivot is provided with a cammed configuration enabling a complex pivot path operative to facilitate passing a connected piercing object through body tissue.
